(19) United States
(12) Patent Application Publication
(10) Pub. No.: US 2021/0353132 A1
(43) Pub. Date: Nov. 18, 2021

(54) ENDOSCOPES AND RELATED METHODS

(71) Applicant: TRITON ENDOSCOPY, LLC, Salt Lake City, UT (US)

(72) Inventors: Dustin Edward Gorringe, Riverton, UT (US); Travis Sessions, Cedar Hills, UT (US); Brian Russell Curtis, Riverton, UT (US)

(73) Assignee: TRITON ENDOSCOPY, LLC, Salt Lake City, UT (US)

(21) Appl. No.: 17/389,930

(22) Filed: Jul. 30, 2021

Related U.S. Application Data
(63) Continuation of application No. PCT/US2020/016294, filed on Jan. 31, 2020.
(60) Provisional application No. 62/799,039, filed on Jan. 31, 2019.

(51) Int. Cl. A61B 1/005

(52) U.S. Cl. CPC A61B 1/0052; A61B 1/307; A61B 1/0057

(57) ABSTRACT

An endoscope can include an elongated shaft that includes a distal tip and a displaceable distal region. The endoscope can further include a handle coupled to the elongated shaft and an actuator movable relative to the handle. The endoscope can further include at least one tensioning line extending through at least a proximal portion of the elongated shaft and through a portion of the handle, and the at least one tensioning line can be coupled to the displaceable distal region of the shaft and also can be coupled to the actuator such that movement of the actuator relative to the handle effects angular movement of the displaceable distal region. The endoscope can further include a locking mechanism that prevents angular movement of the displaceable distal region when the locking mechanism is engaged.

### CROSS-REFERENCE TO RELATED APPLICATIONS

This application is a continuation of International Application No. PCT/US2020/016294, titled ENDOSCOPES AND RELATED METHODS, filed Jan. 31, 2020, which claims priority to U.S. Provisional Patent Application No. 62/799,039, titled ENDOSCOPES AND RELATED METHODS, filed Jan. 31, 2019, the entire contents of each of which are hereby incorporated by reference herein.

### TECHNICAL FIELD

Certain embodiments described herein relate generally to endoscopes and related methods for using the same, and further embodiments relate more particularly to ureteroscopes and related methods for using the same.

### BACKGROUND

Known endoscopes suffer from a variety of drawbacks and/or would benefit from improvements. Embodiments disclosed herein remedy, ameliorate, or avoid one or more of such drawbacks and/or include improvements relative to known devices.

### BRIEF DESCRIPTION OF THE DRAWINGS

The written disclosure herein describes illustrative embodiments that are non-limiting and non-exhaustive. Reference is made to certain of such illustrative embodiments that are depicted in the figures, in which:

FIG. 1 depicts an embodiment of a system that includes a ureteroscope, shown in perspective, and a monitor, shown schematically in elevation;

FIG. 2 is a rear perspective view of an embodiment of a handle of the ureteroscope;

FIG. 3 is a front perspective view of the handle of the ureteroscope;

FIG. 4 is an enlarged perspective view of the handle with a housing portion removed to permit viewing of interior components;

FIG. 5 is an enlarged elevation view of the handle of the ureteroscope when the actuator is in an unactuated state;

FIG. 6 is an elevation view of a displaceable portion of a shaft of the ureteroscope;

FIG. 7 is a perspective view of an embodiment of a cam;

FIG. 8 is a perspective view of an embodiment of a lever;

FIG. 9A is a perspective view of an embodiment of a stopper;

FIG. 9B is a side elevation view of the stopper;

FIG. 9C is a perspective view of an embodiment of a compression spring;

FIG. 10 is a perspective view of an embodiment of an actuator;

FIG. 11 is a perspective view of an embodiment of a housing element, which can cooperate with at least an additional housing element to form a housing of the handle;

FIG. 12 is a perspective view of an embodiment of a distal tip;

FIG. 13 is an enlarged elevation view, similar to that of FIG. 5, of another embodiment of a handle of a ureteroscope that includes a locking actuator in an unactuated state;

FIG. 14 is an elevation view of another embodiment of a system that includes a ureteroscope;

FIG. 15 is a perspective view of an embodiment of a trigger-locking mechanism;

FIG. 16 is a perspective view of an embodiment of a trigger;

FIG. 17 is a perspective view of an embodiment of a trigger lock;

FIG. 18 is a perspective view of an embodiment of a cam-locking mechanism;

FIG. 19A is a perspective view of an embodiment of a cam that is compatible with the cam-locking mechanism of FIG. 18;

FIG. 19B is another perspective view of the cam;

FIG. 20 is a perspective view of an embodiment of a stopper;

FIG. 21 is a perspective view of an embodiment of a linkage;

FIG. 22 is a perspective view of another embodiment of a locking mechanism;

FIG. 23 is a perspective view a proximal end of another embodiment of an endoscope;

FIG. 24 is a perspective view of a proximal end of a housing portion of the endoscope;

FIG. 25 is a perspective view of an embodiment of a lever;

FIG. 26 is a perspective view of an embodiment of a locking element; and

FIG. 27 is a perspective view of another embodiment of a locking element.

### DETAILED DESCRIPTION

The present disclosure relates generally to endoscopes and related methods for using the same. While much of the present disclosure is provided in the context of ureteroscopes, it should be understood that at least some of the features and advantages described herein are applicable to other varieties of endoscopes and/or other elongated medical instruments configured for insertion into the body of a patient. Accordingly, the term “ureteroscope” may, as appropriate, be replaced with the term “endoscope” throughout this specification.

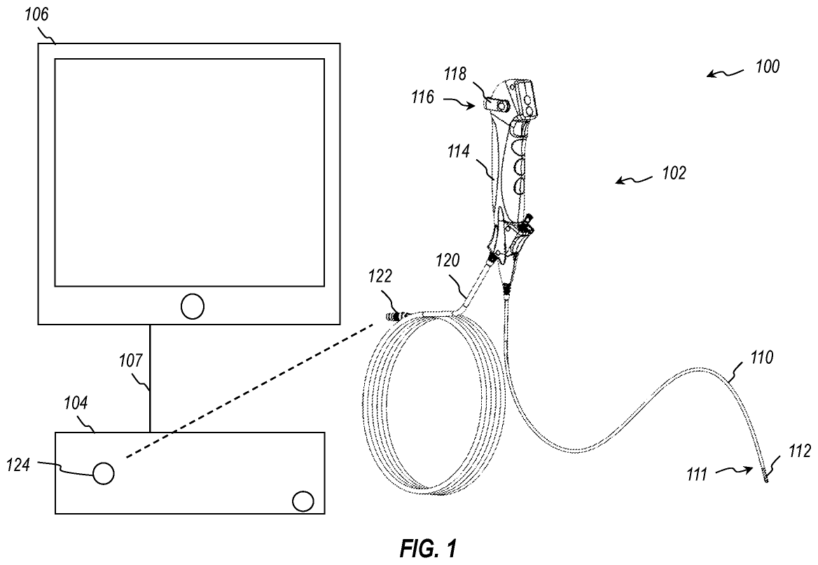

With reference to FIG. 1, in certain embodiments, an endoscope system, or specifically, a ureteroscope system 100, includes a ureteroscope 102, a display interface 104, and a display 106. The ureteroscope 102 is depicted in perspective, while the display interface 104 and the display 106 are shown schematically in elevation view.

The ureteroscope 102 includes an elongated catheter or shaft 110 that can be inserted into or through, or stated otherwise, can navigate through, any suitable regions of the body, such as the urinary tract (the bladder, a ureter, etc.). Other varieties of endoscopes may be configured for insertion into or through, or advancement or navigation through, other suitable regions of the body, as applicable. The ureteroscope 102 can include a tip 112 at a distal end of the shaft 110.

A proximal end of the shaft 110 can be relative stiff, which can be useful for pushability, steering, and/or torqueing as the shaft 110 is advanced within the patient. In various embodiments, a distal portion of the shaft 110 can be configured to be steerable, to deflect, or otherwise to curve or curl, as discussed further below. For example, in some embodiments, the shaft 110 may be selectively curved or angularly displaced through up to 270 degrees in one direction and up to 270 degrees in an opposite direction. Any other suitable deflection angles are contemplated.

The shaft 110 can be attached at a proximal end thereof to a handle 114. The handle 114 can include any suitable actuator 116, such as a lever 118. In the illustrated embodiment, the lever 118 can be moved bidirectionally, such as up or down, relative to the handle 114 to control an angular orientation of the distal tip 112. As further discussed below, the lever 118 can be used to tension tensioning lines that extend through the shaft 110 and into the handle 114, which tensioning lines may also be referred to as control lines, tensioning wires, control wires, etc., to effect rotation of a distal portion of the shaft 110. The actuator 116 may also be referred to as a tensioning actuator 116.

The handle 114 can include any suitable circuitry and/or controls (not shown) that are electrically or otherwise communicatively coupled with the imaging system of the tip 112. The circuitry and/or controls may further be electrically or otherwise communicatively coupled with a cable 120 that is coupled to the handle 114 at a proximal end thereof.

The cable 120 can include any suitable connector 122 or other interfacing mechanism at a distal end thereof. For example, in some embodiments, the cable 120 comprises an HDMI cable, which may be compatible with existing hospital monitors. Any other suitable interface is contemplated. The connector 122 can be coupled to a port 124 of the display interface 104 to permit communication between the circuitry and/or the controls within the handle 114 and the display interface 104. The display interface 104 can include image processing hardware and/or software, one or more controllers, etc.

The display interface 104 can be electrically or otherwise communicatively coupled with the display 106 in any suitable manner, as represented by a communication line 107 in FIG. 1. In some embodiments, the display interface 104 and the display 106 may be integrated into a single unit. The display 106 can display images (e.g., still images or video) obtained via an imaging assembly of the distal tip 112 of the ureteroscope 102. In certain embodiments, the display interface 104 and/or the display 106 can be mounted to a cart, a stand (e.g., a wheeled stand), etc. to achieve, for example, ready maneuverability thereof.

In some embodiments, rather than including certain circuitry and/or controls within the handle 114, the circuitry and/or controls can instead be incorporated into the display interface 104.

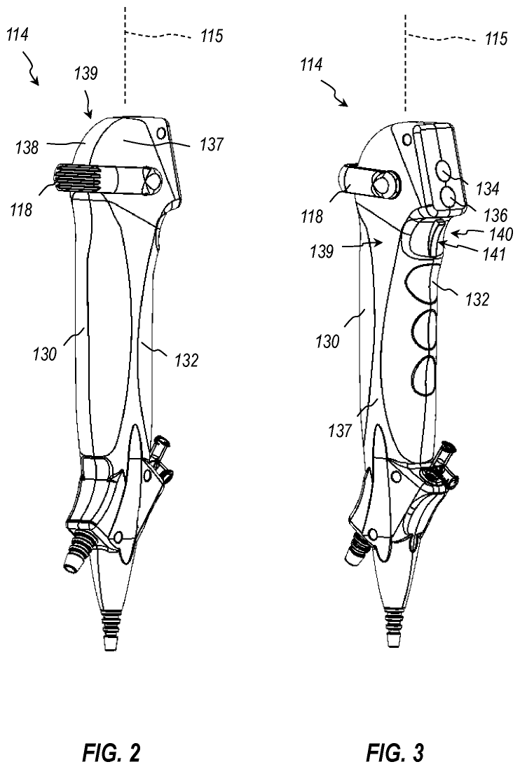

With reference to FIGS. 2 and 3, in the illustrated embodiment, the handle 114 is ergonomically designed to readily be held by a single hand of a practitioner. In some embodiments, the handle 114 includes soft grip regions 130, 132, which can enhance the grippability and improve user feel of the handle 114. In other embodiments, the handle 114 is devoid of soft grip regions, which may, in some instances, provide a preferable feel to practitioners who hold the handle 114 with gloved hands (e.g., who wear latex or other gloves). In the illustrated embodiment, the lever 118 is positioned at an upper or proximal end of the handle 114 and faces rearward so as to be readily engageable by a thumb. Other configurations are contemplated. In use, the handle 114 may be configured so as to remain cool to the touch during a procedure. The handle 114 may also be lightweight.

With reference to FIG. 3, the illustrated embodiment includes a pair of control buttons 134, 136. The control buttons 134, 136 are situated at a front side of the handle 114, and specifically, at the upper or proximal end of the handle 114. The control buttons 134, 136 are positioned substantially opposite the lever 118. The control buttons 134, 136 can be easily accessed, and may be readily engaged or pressed by a finger (e.g., an index finger) of a practitioner while the practitioner grips the handle 114. In some embodiments, a first of the control buttons 134, 136 is used to capture still images that are captured by the imaging assembly at the distal tip 112. In other or further embodiments, a second of the control buttons 134, 136 is used to start and stop a video feed from the imaging assembly. For example, the second of the control buttons may be actuated in a toggle fashion, such that a first press of the button begins capturing video and a second press of the button stops the video capture.

In the illustrated embodiment, each button 134, 136 is actuated by depressing the button 134, 136 substantially in a rearward direction. Stated otherwise, the buttons 134, 136 may be moved substantially toward a longitudinal axis 115 of the handle 114, which is oriented substantially vertically in FIGS. 2 and 3.

With reference to FIGS. 2, 3, and 4, in some embodiments, the handle 114 may include a pair of housing elements 137, 138 that are joined together to form a housing 139, or a housing portion of the handle 114. In the illustrated embodiment, the housing elements 137, 138 are elongated pieces that extend substantially longitudinally. The soft grips 130, 132 may be positioned over the housing elements 137, 138 or otherwise joined thereto.

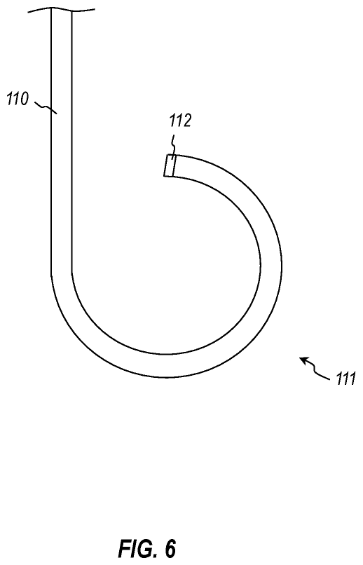

With reference to FIGS. 3, 4 and 5, in some embodiments, the handle 114 includes a locking mechanism 140, which can prevent angular movement of a displaceable distal region of the shaft 110 when the locking mechanism 140 is engaged. For example, with reference again to FIG. 1, and with additional reference to FIG. 6, the shaft 110 can include a distal region 111 that is angularly adjustable, or stated otherwise, that can be steered, guided, curved, or otherwise angularly transitioned so as to reorient the distal tip 112. In various embodiments, a range of angular movement of the distal region 111 can be from about −270 degrees to about +270 degrees, or stated otherwise, of up to about 270 degrees in either of two substantially opposite directions. Stated otherwise, an angular orientation of the distal tip 112 can be adjusted through a series of angles, which in some embodiments, yield a maximum angular displacement of up to 270 degrees in either direction. Other maximum angular displacements are also contemplated. The locking mechanism 140 may also be referred to as a cam-locking mechanism, an articulation-locking mechanism, or an articulation-locking trigger.

With reference again to FIGS. 3, 4, and 5, in the illustrated embodiment, the locking mechanism 140 includes a locking actuator 141, such as a trigger 142 coupled with a stopper 144. The trigger 142 may also or alternatively be referred to as a button, etc. The stopper 144 can additionally or alternatively be referred to as a stop, wedge, engagement mechanism, etc. As further discussed below, the stopper 144 can engage a tensioning cam to prevent rotation thereof. More generally, the stopper 144 may be referred to as a coupling, link, or linkage 191. In the illustrated embodiment, the linkage 191, which also functions as a stopper in the present embodiment, transfers movement of the trigger 142 to the tensioning cam. In particular, depression of the trigger 142 causes the linkage 191 to move inwardly and into contact with the tensioning cam.

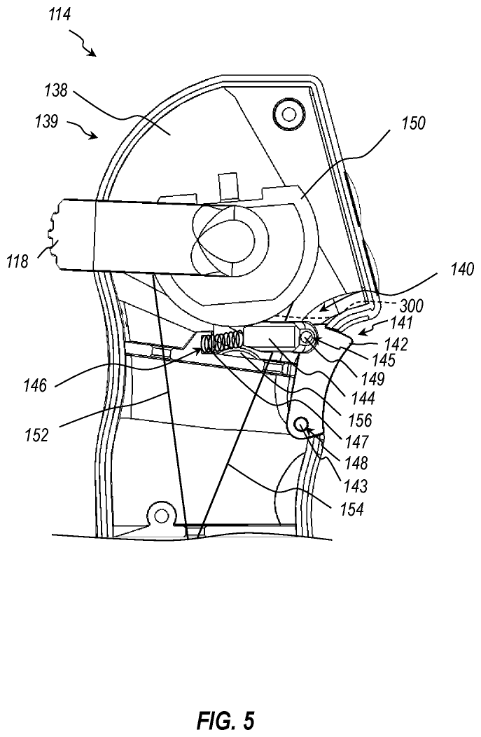

The trigger 142 may be coupled with the stopper 144 in any suitable manner. In the illustrated embodiment, the trigger 142 is pivotably coupled with the stopper 144 via a pivot member 149 (FIG. 5). The pivot member 149, may take any suitable form, such as, for example, a cylindrical rod that extends transversely through an opening 145 defined by the trigger 142 (see also FIG. 10). The pivot member 149 can, for example, be fixedly secured to one of the stopper 144 and the trigger 142, and the other of the stopper 144 and the trigger 142 can be freely rotatable about the pivot member 149. Other mounting configurations are contemplated.

For example, the trigger 142 and/or the stopper 144 may instead linearly translate within a track defined by the handle 114. In some instances, the trigger 142 and the stopper 144 may be fixedly secured to one another. In still further embodiments, the trigger 142 and the stopper 144 are integrally formed from a unitary piece of material as an integral, unitary component.

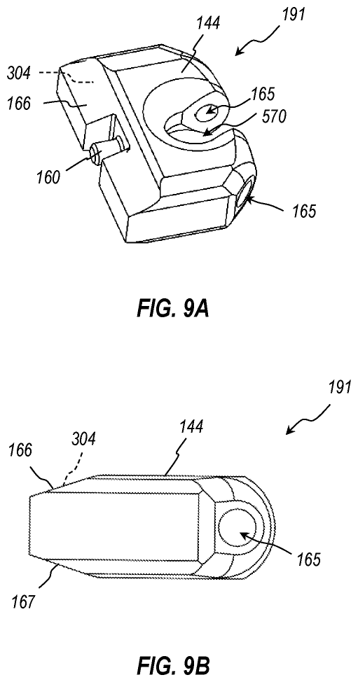

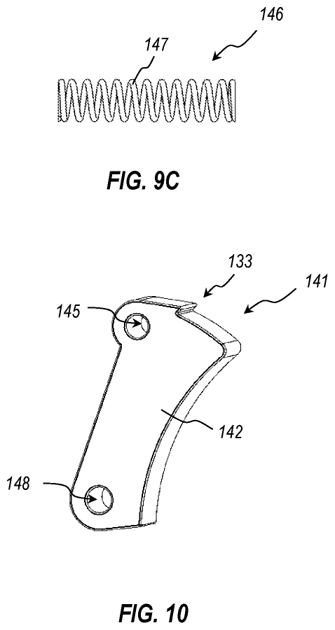

In the illustrated embodiment, the locking mechanism 140 further includes a biasing member 146, such as a compression spring 147 (see also FIG. 9C), that biases the locking mechanism 140 to an unactuated or disengaged state. In the illustrated embodiment, the trigger 142 is positioned substantially opposite the lever 118. In some embodiments, the trigger 142 can be pressed and held by a finger (e.g., the index finger) of the hand in which the practitioner is holding the handle 114. In some embodiments, the trigger 142 may advantageously be engaged at the same time as the lever 118 and/or shortly after releasing the lever 118, and the simultaneous and/or near simultaneous actuation may be accomplished with a single hand.

In the illustrated embodiment, the locking actuator 141 is transitioned into a locked orientation by moving the trigger 142 substantially in the direction of the lever 118. Engagement of the lever 118 and the trigger 142 may be accomplished by different fingers of the same hand, and may be accomplished simultaneously or serially. Stated otherwise, the lever 118 can be engaged with one finger (e.g., the thumb) of a hand that holds the handle 114 and, in some instances, a component of the force applied by that finger as it actuates the lever (e.g., up or down) can be directed toward the longitudinal axis 115. The trigger 142 can be engaged with a different finger (e.g., the index finger) of the same hand and, in general, a primary component of the force applied by that finger to transition the trigger 142 to the locked orientation can be directed toward the longitudinal axis 115. The components of force that are directed toward the longitudinal axis 115 in actuating the lever 118 and the trigger 142 can be directed substantially opposite to one another (e.g., may be parallel to one another and each directed radially toward the longitudinal axis 115). In general, it may be said that the trigger 142 can be actuated by applying force thereto substantially rearwardly or toward the longitudinal axis 115. The arrangement of the trigger 142 can be ergonomic and used intuitively by a practitioner.

In the illustrated embodiment, the trigger 142 is mounted to the handle 114 so as to be rotational about a further pivot member 143. Stated otherwise, the trigger 142 is pivotably mounted to the handle 114, or more specifically, to the housing 139. The pivot member 143, may take any suitable form, such as, for example, a cylindrical rod that extends transversely through an opening 148 defined by the trigger 142 (see also FIG. 10). The pivot member 143 can be fixedly secured to one of the housing 139 and the trigger 142. The trigger 142 may, for example, rotate about the fixed pivot member 143 as the trigger 142 is actuated, or the pivot member 143 may rotate relative to the housing 139 as the trigger 142 is actuated. Other mounting configurations are contemplated. For example, as previously mentioned, in some embodiments, the trigger 142 instead linearly translates inward (e.g., when depressed) and outward (e.g., when released) within a track defined by the handle 114.

The locking mechanism 140 may be said to include and/or may be said to interact with a cam 150 to which one or more tensioning lines 152, 154 are secured. The cam 150 may also be referred to as a wheel or tensioning hub. The one or more tensioning lines 152, 154 extend through at least a portion of the handle 114 (e.g., through at least central and distal regions of the housing 139) and through at least a proximal portion of the elongated shaft 110. The one or more tensioning lines 152, 154 are coupled to the displaceable distal region 111 of the shaft, and are also coupled to the actuator 116 (lever 118)—in the illustrated embodiment, the lever 118 is fixedly secured to the cam 150—such that movement of the actuator 116 relative to the handle 114 effects angular movement of the cam 150. As the cam 150 is thus moved angularly, it pushes or allows slack on one of the tensioning lines 152, 154 and pulls on the other of the tensioning lines 152, 154, depending on the direction the actuator 116 is moved. This movement of the tensioning lines 152, 154 results in angular movement of the displaceable distal region 111.

Depressing the trigger 142 can urge the stopper 144 rearward so as to wedge the stopper 144 between the cam 150 and one or more protrusions 156 defined by one or more of the housing elements 137, 138. This can lock the cam 150 into a fixed angular position relative to the housing 139, or stated otherwise, relative to the handle 114. As a result, the tensioning lines 152, 154 can be locked in place relative to the handle 114, thus maintaining an angular orientation of the distal region 111 of the shaft 110.

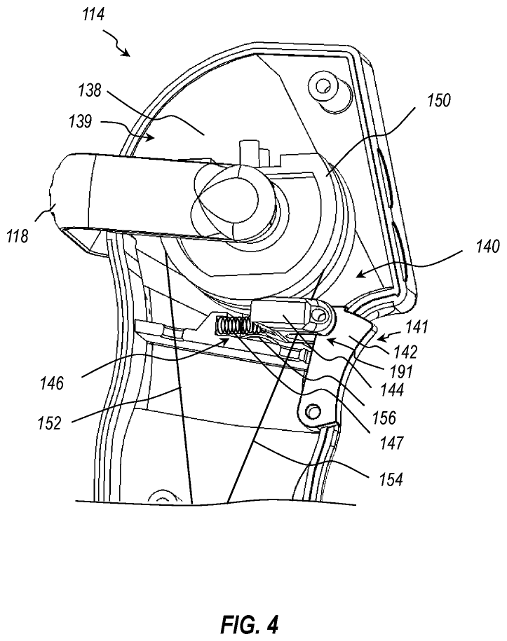

With continued reference to FIGS. 4 and 5, the biasing member 146 can provide a continual bias in the forward or rightward (in the illustrated orientation) direction. To depress the trigger 142, the practitioner must overcome the bias to urge the stopper 144 rearwardly (leftward, in the illustrated orientation) into frictional engagement with the cam 150. Moreover, the practitioner must continually overcome the bias to maintain the stopper in the rearward orientation, or stated otherwise, to keep the stopper in the locked configuration. Upon release of the trigger 142 by a practitioner, the biasing member 146 automatically urges the stopper 144 out of frictional engagement with the cam 150, thus permitting the cam 150 to move freely, whether by further movement of the lever 118 and/or by straightening of the distal region 111 of the shaft 110, such as may occur due to contact between the distal region 111 of the shaft and anatomical structures of the patient as the ureteroscope 102 is removed. In the illustrated embodiment, a first end of the spring 147 is retained within a recess cooperatively defined by the housing elements 137, 138, and a second end of the spring is mounted on a post 160 defined by the stopper 144 (see FIG. 9A).

In the illustrated embodiment, the locking mechanism 140 is selectively engageable. Stated otherwise, a user can select whether or not to engage the locking mechanism 140, and can engage the locking mechanism 140 by pressing on the trigger 142. Moreover, in the illustrated embodiment, the locking mechanism 140 permits for variable friction to be applied to the cam 150. That is, greater friction can be achieved by applying greater force to the trigger 142.

In other embodiments, the locking mechanism 140 may be automated or automatic. For example, in some embodiments, the locking mechanism 140 may naturally engage one or more of the cam 150 or the lever 118 at any point of movement of the lever 118. For example, the cam 150 may be frictionally mounted within the housing 139 such that the cam 150 naturally remains fixed at whatever angular orientation to which it is moved via the lever 118. In certain of such embodiments, a practitioner would manually move the lever 118 to an unactuated position to straighten out the distal region 111 prior to removal of the shaft 110 from the patient.

In other or further embodiments, the locking mechanism 140 may interact directly with the lever 118 to maintain the lever 118 in a desired position and, hence, the distal region 111 of the sheath 110 in a desired angular orientation. For example, in some embodiments, the locking mechanism 140 is configured to contact and lock the lever 118 into a fixed orientation relative to the housing 139, as further described with respect to certain embodiments below.

In other or further embodiments, the locking mechanism 140 directly locks the cam 150 in place. In certain of such embodiments, the locking mechanism 140 must be selectively deactivated by a user to permit the distal region 111 of the sheath 110 to return to a neutral or linear state. For example, in some embodiments, the cam 150 may include a series of holes that extend transversely therethrough, and the locking mechanism can urge one or more posts or pins through the holes to maintain the cam 150 in a fixed angular orientation relative to the housing 139. The locking mechanism can be selectively deactivated to remove the posts or pins from the cam 150, thereby freeing the cam 150 to again rotate relative to the housing 139. In other embodiments, the arrangement of posts and holes may be reversed.

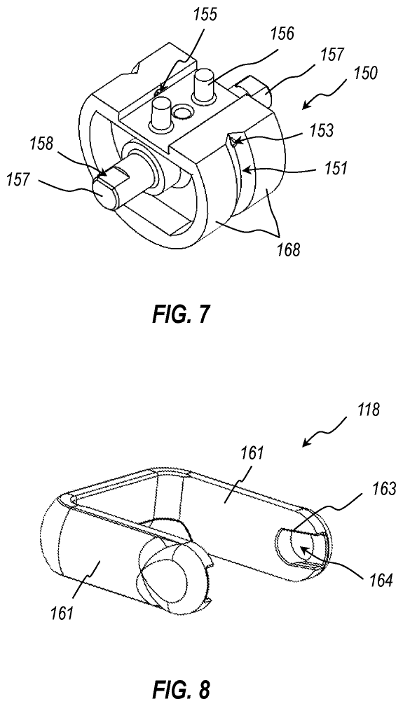

With reference to FIGS. 7-11, further details of various components of and/or that are associated with the handle 114 are provided. With respect to FIG. 7, in some embodiments, the cam 150 includes a channel or groove 151 that extends about a lower end of the cam 150. The groove 151 can be configured to receive therein a portion of each tensioning line 152, 154. As the cam 150 rotates, portions of the tensioning lines 152, 154 can generally roll over the cam 150 within the groove 151, which can help to maintain an alignment of the tensioning lines 152, 154.

In some embodiments, the cam 150 can include a channel 153 and a channel 155 at opposite ends of the groove 151. A separate tensioning line 152, 154 can extend through a respective one of the channels 153, 155. The tensioning lies 152, 154 can be secured to one or more posts 156 in any suitable manner. Other securement arrangements for fixing the tensioning lines 152, 154 to the cam 150 are contemplated.

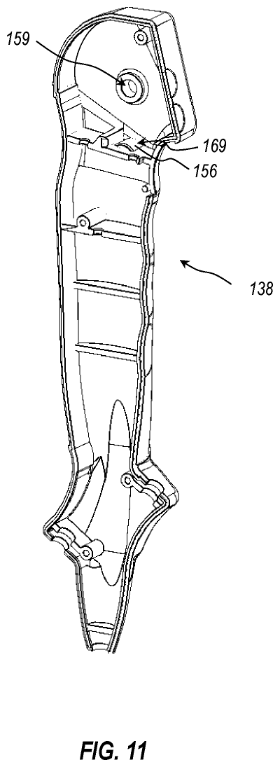

In some embodiments, the cam 150 includes a pair of rods 157 that extend transversely from a body of the cam 150. The rods 157 can extend through a respective opening in each housing member 137, 138. For example, one of the rods 157 can extend through an opening 159 in the housing member 138 (see FIG. 11), and the other rod 157 can extend through a similar opening in the housing member 137. The rods 157 can be fixedly secured to the lever 118, as discussed below with respect to FIG. 8. In the illustrated embodiment, each rod 157 includes any suitable keying feature 158, such as a notch or a flat, that is configured to establish and/or maintain a fixed angular orientation between the cam 150 and the lever 118.

With reference to FIG. 8, in the illustrated embodiment, the lever 118 includes a pair of arms 162. Each arm 162 includes a recess 163 at its end and at an inner region thereof. Each recess 163 is configured to receive an end of one of the rods 157. In the illustrated embodiment, each recess 163 includes a keying feature 164 configured to cooperate with the keying feature 158 of the cam 150 to establish and/or maintain the fixed angular orientation between these components. In the illustrated embodiment, the keying feature 164 comprises a flat region at an end of a semi-circular region.

In use, the lever 118 can be rotated up or down relative to the handle 114. This up or down movement rotates the cam 150 in tandem. In the illustrated embodiment, the rods 157 of the cam extend outwardly through the openings 159 in the housing members 138, 139, and rotate within those openings 159. The lever 118 remains entirely at an exterior of the handle 114. In other embodiments, the lever 118 may include one or more inward protrusions or rods that extend inwardly through one or more of the housing members 138, 139 to couple with the cam 150. In some embodiments, an entirety of the cam 150 is positioned at an interior of the handle 114.

With reference to FIGS. 9A and 9B, in the illustrated embodiment, the stopper 144 includes a channel 165 through which the pivot member 149 is received. The cannel 165 aligns with the opening 149 of the trigger 142.

The stopper 144 can include a cam-engaging surface 166 and a housing-engaging surface 167. More generally, these may be referred to as engagement surfaces 166, 167. In the illustrated embodiment, the engagement surfaces 166, 167 are angled (e.g., define an acute angle therebetween) so as to define a wedge shape. In the illustrated embodiment, the engagement surfaces 166, 167 are formed of the same material as the remainder of the stopper 144. Stated otherwise, in some embodiments, the stopper 144 and the engagement surfaces 166, 167 are formed as a unitary component.

In some embodiments, one or more of the engagement surfaces 166, 167 can include an engagement-enhancing feature which may be integral with or attached to the stopper 144. The engagement-enhancing feature may be one or more of a friction-enhancing member or an interlocking member. Suitable friction-enhancing members can include, for example, one or more of surface treatments (e.g., scuffs, scratches, bumps, knurling, grooves, high-friction coatings) or friction pads (e.g., pads formed of rubber or other high-friction material). Suitable interlocking members include, for example, teeth or gears.

With reference again to FIG. 7, the cam 150 can include an engagement surface 168 configured to interface with the engagement surface 166 of the stopper 144. The protrusion 156 of the housing can include an engagement surface 169 (see FIG. 11) configured to interface with the engagement surface 167 of the stopper 144.

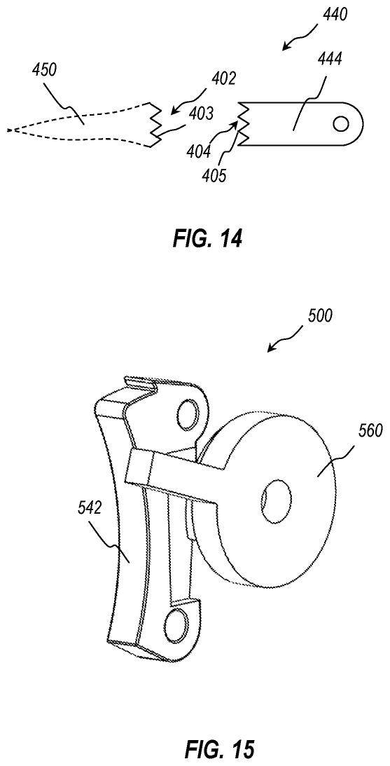

Any suitable combination of the engagement surfaces 166, 167, 168, 169 can include a friction-enhancing member. For example, in various embodiments, one or both of the engagement surfaces 166, 168 of the stopper 144 and the cam 150, respectively, can include a friction-enhancing member. In some embodiments, the engagement surface 166 of the stopper 144 includes a series of interlocking members, such as teeth, and the engagement surface 168 of the cam 150 includes complementary interlocking members, such as teeth that are configured (e.g., sized and/or oriented) to interlock with the teeth of the stopper 144. One example of such an arrangement is discussed below with respect to FIG. 14. Any suitable engagement interfaces among the various components are contemplated.

With reference to FIG. 10, in some embodiments, the trigger 142 can include a catch 133 that is configured to cooperate with the housing to maintain a portion of trigger 142 within the housing against the bias provided by the spring 147. Other features of the trigger 142 that are identified in FIG. 10 were previously discussed.

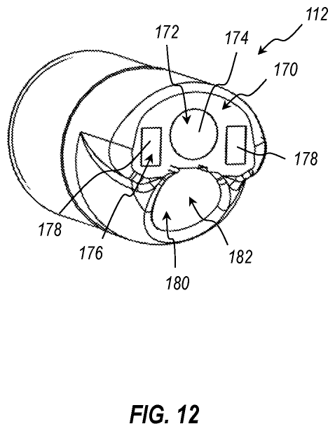

With reference to FIG. 12, the tip 112 can include an imaging system 170, which may also be referred to as an imaging assembly. The imaging assembly 170 can include an image-gathering system 172, of which at least a portion is positioned so as to directly receive imaging data from beyond a distal tip of the sheath 110 when the sheath 110 is inserted into the patient. Stated otherwise, at least a portion of the image-gathering system 172 can be oriented relative to the sheath 110 so as to observe a region distal to the distal tip 112 of the sheath 110. In the illustrated embodiment, the image-gathering system 110 includes an imaging device 174 positioned at (e.g., embedded within) the distal tip 112.

Any suitable imaging device 174 is contemplated. For example, in the illustrated embodiment, the imaging device 174 includes an image sensor of any suitable variety, including those presently available and those yet to be devised. The image sensor may also be referred to as an imaging sensor or as an imager. The image sensor can, for example, comprise any suitable sensor that detects information used to make an image, which image can be a visual representation of the physical region detected by the sensor. Illustrative examples of suitable image sensors include analog and/or digital varieties of one or more of camera or video sensors, such as, for example, semiconductor charge-coupled devices or active pixel sensors of any suitable variety (e.g., complementary metal-oxide-semiconductor [CMOS], N-type metal-oxide-semiconductor [NMOS or Live MOS]). In other or further applications, suitable sensors may include thermal imaging sensors (e.g., infrared sensors), ultrasound sensors (e.g., ultrasonic transducers), etc. In some embodiments, the image sensor may be configured to convert an observed or detected phenomenon (e.g., light, infrared radiation, sound) into an analog or digital electrical signal representative of the observed phenomenon. In other embodiments, the image sensor may be configured to capture and/or directly transport the observed phenomenon. For example, in some embodiments, the image sensor may comprise an input end of a waveguide, such as an optical fiber or optical fiber bundle, which may capture and transport one or more signals away from the distal end of the sheath. In various embodiments, the image sensor can include one or more lenses or lens systems.

In the illustrated embodiment, the imaging device or image sensor 174 comprises a camera sensor configured to convert visible light into electrical signals that are representative of the detected light for transport ureteroscope 102. In some embodiments, the image sensor 174 comprises a CMOS camera, which in further embodiments, may comprise a 400×400 resolution, or 160,000 pixels. The camera may have a wide field of view, such as, for example, up to 120 degrees.

The image sensor 174 can be electrically coupled with one or more communication lines via which the electrical signals are transported through or along the sheath. The one or more communication lines may extend through at least a portion of the sheath 110.

The imaging assembly 170 can further include a lighting assembly 176 that is positioned at (e.g., is embedded within) the distal tip 112. In the illustrated embodiment, the lighting assembly 176 includes a pair of cool LEDs or LED assemblies 178 at opposite sides of the image sensor 174. In some embodiments, operational controls of the LEDs are configured to auto-adjust an amount of light provided by the LEDs, such as to optimize the image quality obtainable by the image sensor 174.

The tip 112 can further include an exit port 180 of a working channel 182. The working channel 182 can extend through the shaft 110. In some embodiments, the working channel 182 is relatively large and can be configured to accommodate the passage of large devices therethrough, such as, for example, laser probes, electrohydraulic lithotripsy probes, radiofrequency ablation probes, etc. In various embodiments, the tip 112 can include atraumatic rounded edges.

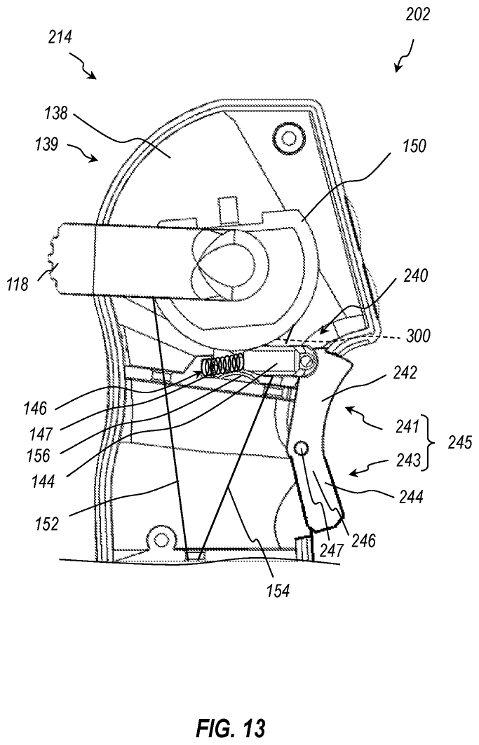

FIG. 13 depicts a proximal portion of another embodiment of a handle 214 of another embodiment of a ureteroscope 202. Many of the components of the handle 214 are identical to those discussed above with respect to FIG. 5 and thus are numbered identically. The handle 214 includes a locking mechanism 240, however, that differs from the locking mechanism 140 in certain respects.

The locking mechanism 240 includes the stopper 144 that includes a wedge-shaped portion, which can selectively frictionally engage the cam 150 in manners such as previously disclosed. The cam 150, however, is coupled with a different actuator 245.

The actuator 245 includes both a locking actuator 241, which is similar to the locking actuator 141 described above, and also a release actuator 243. The release actuator 243 can be manually actuated to selectively move the stopper 144 from an engaged state, relative to the cam 150, to a disengaged state.

In the illustrated embodiment, a single actuation member 246 comprises both the locking actuator 241 and the release actuator 243. Stated otherwise, the locking actuator 241 and the release actuator 243 are integrally formed of a unitary piece of material. The actuation member 246 is pivotally coupled to the housing 139 so as to selectively rotate in either direction about a pivot 247.

The actuator member 246 includes a trigger portion 242, which resembles the trigger 142 discussed above, and further includes a release member 244. The release member 244 is defined by a lower end of the actuator member 246.

In use, a practitioner can depress the trigger portion 242 to overcome a bias provided by the spring 147 and to urge the wedge portion of the stopper 144 into frictional engagement with an outer surface of the cam 150—specifically, to frictionally engage the concavely rounded outer periphery of the cam 150 to prevent rotation of the cam 150 relative to the housing 139.

Upon release of the trigger portion 242, the spring 147 generally provides sufficient biasing force to urge the stopper 144 forwardly out from its engagement with the cam 150 to thereby release the cam 150 and permit the cam 150 to rotate freely relative to the housing 139, such as when actuated by the lever 118. In the event that the stopper 144 were to be subjected to sufficient engagement force so as to get stuck in the engaged position, the spring 147 were to break, and/or some other event occurred that caused the spring 147 to fail to automatically return the stopper 144 to the disengaged state, it could be difficult to manually retract the trigger 242 outwardly relative to the housing 139 to disengage the stopper 144.

The release member 244 can act as a failsafe to permit a user to manually disengage the stopper 144. Stated otherwise, in the event that the stopper 144 becomes stuck in the engaged position, the release member 244 can manually be depressed, thus causing the actuator member 246 to rotate about the pivot 147 and urge the stopper 144 from its engagement with the cam 150.

In other embodiments, the locking actuator 241 and the release actuator 243 can be separate, rather than integrally connected. For example, in some embodiments, the locking actuator 241 is positioned at a front side of the handle 214 in a manner such as that depicted in FIG. 5, and the release actuator 243 is positioned at a rear side of the handle 214. In certain of such embodiments, rather than pulling on the front end of the stopper 144 to urge the stopper out of frictional engagement with the cam 150, the release actuator 243 can push forwardly on the stopper, to urge the stopper out of frictional engagement with the cam 150.

For example, in some embodiments, a forward end of the release actuator 243 can abut a rearward end of the locking actuator 241. In other embodiments, the locking actuator 241 and the release actuator 243 may be integrally formed of a unitary piece of material as a single component. The component can be urged rearwardly via the locking actuator 241 to engage the cam 150, and the component can be urged forwardly via the release actuator 243 to release the cam 150. In some embodiments, the unitary component that includes both actuators 241, 243 extends fully through the handle 114. The locking actuator 241 can, for example, be positioned at the front of the handle 114 and the release actuator 243 can be positioned at the back of the handle 114. Any other suitable arrangement is contemplated.

In some embodiments, the triggers 142, 242 must be continuously depressed to continually overcome the bias provided by the spring. The locking mechanism 140, 240 can automatically return to the unlocked state immediately upon release of the trigger 142, 242. In other embodiments, the trigger 142, 242 may be selectively locked in the actuated or depressed state. For example, in some embodiments, a rotational wedge between the trigger and the housing may be used to selectively lock and unlock the trigger 142, 242 in the depressed state against the bias of the spring. Any other suitable locking mechanism for the trigger 142, 242 is contemplated.

Other or further embodiments include a variety of other or further locking mechanisms to achieve a fixed angular orientation of the distal region 111 of the shaft 110. Any suitable combination of features among the various embodiments disclosed previously or hereafter is contemplated.

For example, with reference again to FIGS. 5 and 13, in some embodiments, either locking mechanism 140, 240 can include a cam 150 that includes a friction-enhancing member 302, such as a friction pad or frictional surface treatment, on its the outer surface (e.g., the rounded cylindrical surface). The friction-enhancing member 302 is identified as the outer surface of the cam 150 in each of FIGS. 5 and 13 by way of a broken line. The broken line indicates that the friction-enhancing member 302 may be absent in some embodiments. Variable friction can achievable in certain embodiments that include the friction-enhancing member 302, as previously discussed. In some embodiments, the trigger 142, 242 can be spring-loaded, such as, e.g., for biasing the trigger 142, 242 to an unactuated or disengaged state.

In some embodiments, the stopper 144 includes a friction-enhancing member 304, as depicted in FIGS. 9A and 9B. The friction-enhancing member 304 can include a friction pad (e.g., a pad of rubber or other high-friction material) or a frictional surface treatment. When the trigger mechanism is actuated (e.g., pulled) the friction pad is pushed into the cam 150, giving rise to friction between the pad and the cam that prevents rotation of the cam 150 in either direction. In other or further embodiments, the cam 150 includes the friction-enhancing member 302 in addition to or instead of the friction-enhancing member 304 of the stopper 144.

With reference to FIG. 14, in some embodiments, a locking mechanism 440 can include a cam 450 and a stopper 444 that are similar to like-numbered features discussed above, with leading numeral having been replaced with the number “4.” Only a portion of the cam 450 is depicted in FIG. 14—in particular, only small angular segment of the generally cylindrical cam 450 is shown. The cam 450 includes an interlocking member 402, which includes a series of teeth 403. The teeth 403 are positioned on the outer surface (e.g., the rounded cylindrical surface) of the cam 450.

The stopper 444 likewise can include an interlocking member 404, which includes a series of teeth 405 that are complementary to the teeth 403. In the illustrated embodiment, each of the teeth 403, 405 has a substantially triangular profile. The teeth 405 may be positioned on an innermost surface of the stopper 444, in some embodiments.

A trigger, such as, for example, either of the triggers 142, 242 discussed above, can be coupled with the stopper 444 in manners such as previously disclosed. For example, in some embodiments, the trigger 142, 242 may be spring-loaded to bias the trigger 142, 242 to the unactuated state. Actuation of the trigger 142, 242 can urge the stopper 444 inwardly (e.g., leftward, in the illustrated orientation) and into engagement with the cam 450. In particular, the teeth 405 may interface, interlock, or interdigitate with the teeth 403 of the cam 450. Once interlocked, the teeth 403, 405 can prevent rotation of the cam 450 relative to the housing.

The locking mechanism 440 can be a binary system, rather than a variable friction system. Stated otherwise, the interlocking members 402, 404 may function in either an engaged state or a disengaged state. There are generally not different or variable levels of engagement, although there may be some amount of unintended or ancillary rotation of the cam 450 as the interlocking members are coming into engagement and aligning with each other. In some instances, employing relatively smaller and/or more numerous teeth can reduce the possibility and/or severity of trigger-action-induced rotation of the cam 450. In some instances, relatively larger teeth may require less trigger force to lock the cam 450 or to maintain the cam 450 in the locked state. In further instances, these and/or other characteristics of differently sized teeth may be balanced to achieve a desired engagement force with minimal ancillary cam rotation.

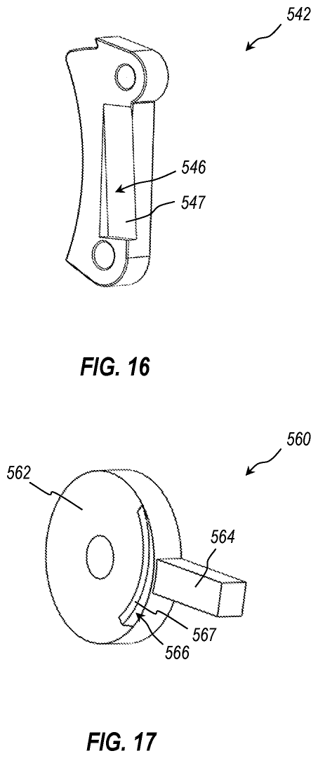

With reference to FIG. 16, generally, in various embodiments, the trigger can be selectively locked in place relative to the handle, such that a practitioner need not continually hold the trigger to maintain a fixed deflection angle of the distal end of the shaft. In various embodiments, the locking mechanism likewise can selectively release the trigger from the actuated state to the unactuated state. Any suitable locking mechanism for maintaining the trigger in an actuated orientation and/or releasing the trigger from the actuated orientation is contemplated. Any of the systems previously disclosed and disclosed hereafter may include a trigger-locking mechanism.

FIG. 16 specifically depicts in illustrative embodiment of a trigger-locking mechanism 500. The trigger-locking mechanism 500 includes a trigger 542, which can resemble the triggers 142, 242 discussed above, and further includes a trigger lock 560. In some instances, the trigger lock 560 may be referred to as a rotational wedge. As further discussed below, the trigger lock 560 can be rotated in a first direction to lock the trigger 542 into a fixed position relative to the housing (such as the housing 139 discussed previously), and can be rotated in the opposite direction to disengage from the trigger 542 and release the trigger 542 from the locked orientation.

With continued reference to FIG. 16, in the illustrated embodiment, the trigger 542 includes a wedge-shaped recess 546. An angled engagement surface 547 slopes inwardly from a side surface of the trigger 542 to define the recess 546.

With reference to FIG. 17, the trigger lock 560 includes a disk 562 that is rotationally mounted to the housing. An actuator 564 projects radially outwardly from an outer edge of the disk 562 and is accessible by a finger of a practitioner. The practitioner can engage the actuator 564 to rotate the trigger lock 560, as desired. The actuator 564 may also be referred to as a trigger-locking actuator or as a lock-locking actuator. The trigger lock 560 further includes a wedge-shaped projection 566 that includes an angled engagement surface 567 that slopes away from a side surface of the disk 562.

With continued reference to FIGS. 16 and 17, and as can be appreciated from the operational configuration of the trigger 542 and the trigger lock 560 depicted in FIG. 15, illustrative use of the trigger-locking mechanism 500 will now be described. The trigger 542 and the trigger lock 560 can be coupled with a housing (e.g., the housing 139) so as to be substantially fixed in a lateral direction, while being permitted to rotate relative to the housing. For example, The trigger 452 and the trigger lock 560 may each be capable of rotating relative to the housing about axes that are substantially parallel to one another, yet the trigger 452 and the trigger lock 560 can be restrained from translating in a transverse direction along either of their respective axes of rotation.

In the illustrated embodiment, the actuator 564 is in a downward orientation such that the engagement surfaces 547, 567 are spaced from each other, disengaged, or not in contact with each other. In this unlocked orientation, the trigger 542 can be readily depressed, as desired, to engage and lock the cam (e.g., the cam 150) and the tensioning lines (e.g., the tensioning lines 152, 154) in manners such as previously disclosed. In certain embodiments, the system can include a biasing member that biases the trigger 542 to the unactuated state, and the trigger 542 can be automatically urged back to the unactuated state under the influence of the biasing member upon release of the trigger 542.

If, on the other hand, the practitioner wishes for the trigger 542 to remain in the actuated state, the trigger 542 can be locked in the actuated state, which may also be referred to a locked state or as a holding state. In particular, the actuator 564 of the trigger lock 560 can be rotated upward while the trigger 542 remains depressed in the actuated state. The upward rotation of the trigger lock 560 can cause the engagement surfaces 547, 567 to frictionally engage one another by an amount sufficient to prevent either the trigger 542 or the trigger lock 560 from moving relative to the housing. For example, due to the inability of the trigger 542 and the trigger lock 560 to move laterally relative to the housing, the frictional engagement of the engagement surfaces 547, 567 can be sufficient to bind the trigger 542 and the trigger lock 560 from further rotational movement relative to each other and relative to the housing. Accordingly, the trigger 542 can remain in a fixed position relative to the housing, even after the practitioner releases the trigger 542. With the trigger 542 in the locked state, the trigger 542 may remain depressed sufficiently to engage and fix the tensioning cam (e.g., the cam 150) relative to the housing (e.g., the housing 139), and thus a deflection angle of the distal region of the shaft can be held in place.

Downward rotation of the actuator 564 can disengage the engagement surfaces 547, 567 and thereby unlock the trigger 542. That is, when desired, a practitioner may unlock the trigger 542 by rotating the actuator 564 in the opposite direction. Other locking and unlocking mechanisms are contemplated.

Various dimensions of the trigger lock 560 can be adjusted to ensure the trigger 542, the trigger lock 560 are able to function together in the manner described. For example, some systems may additionally include a stopper, such as in the arrangement depicted in FIGS. 4 and 5. In certain of such embodiments, the disk 562 may be thinner than shown in FIGS. 15 and 17 and/or have a smaller outer diameter. In other or further instances, a span or recess 570 (see FIG. 9A) of the stopper 144, into which a portion of the trigger 542 is received, may be increased so as to additionally accommodate the disk 562. In other embodiments, the recess 546 and protrusion 566 of the trigger 542 and the trigger lock 560, respectively, can be reversed.

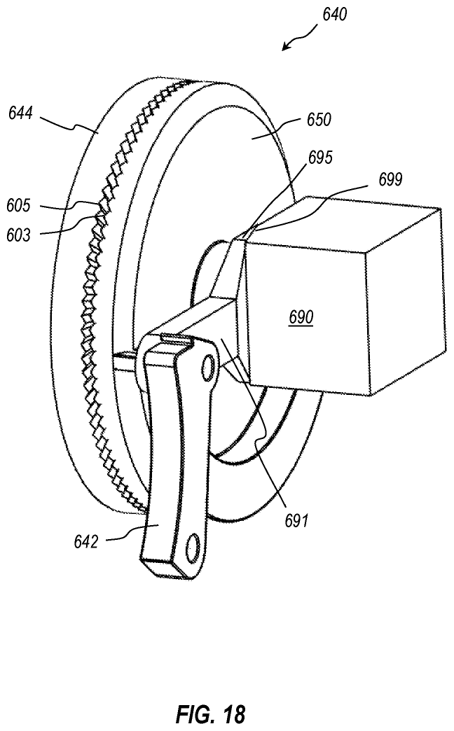

With reference to FIG. 18, in general, in some embodiments, a lateral or side surface of a tensioning hub or cam can be engaged to fix the cam relative to the housing, rather than (or in addition to) an outside or circumferential surface thereof. In some embodiments, the engagement may be frictional, and may yield a fixation of variable strength. In other embodiments, such as depicted in FIG. 18, the engagement may be substantially binary, such as may result from the engagement of complementary sets of teeth.

In particular, FIG. 18 depicts an illustrative embodiment of a cam-locking mechanism 640 that can be used with any suitable handle and housing configuration, such as the housing 139 discussed above. The cam-locking mechanism 640 includes a tensioning cam 650, which may resemble other cams disclosed herein. The cam-locking mechanism 640 further includes a trigger 642, which can be substantially the same as the trigger 142. Indeed, as with other triggers disclosed herein, the trigger 642 may be referred to as a locking actuator. The trigger 642 can be pivotably coupled with a linkage 691 via which the trigger 642 can interact with the cam 650, as further discussed below. The mechanism 640 further includes a stopper 644 and a deflection member 690.

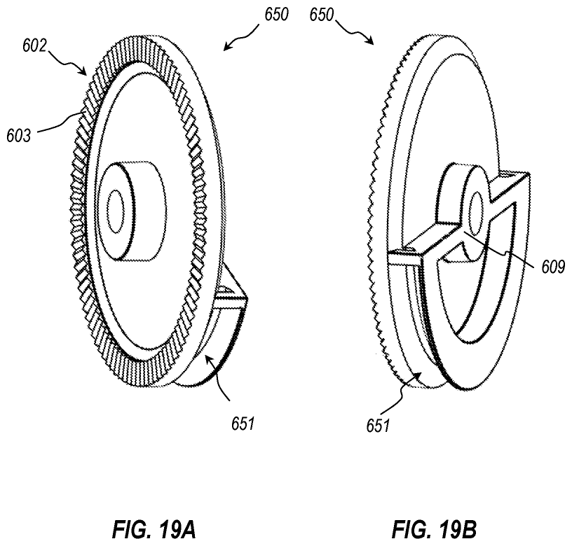

With reference to FIGS. 19A and 19B, the cam 650 can be securely fastened to tensioning lines, which can be received within a portion of a channel 651. The cam 650 can further include an interlocking member 602, which includes a series of teeth 603 that extend laterally from a lateral face of the cam 650. In the illustrated embodiment, the teeth 603 are positioned about a full periphery of the cam 650. The cam 650 can further include a deflection surface 609.

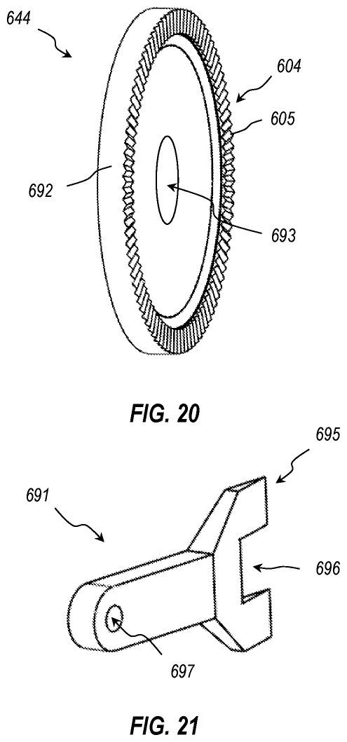

With reference to FIG. 20, the stopper 644 can include a disk 602 that defines an interlocking member 604 that is substantially complementary to the interlocking member 602. The interlocking member 604 can define a series of laterally extending teeth 605 that face toward the teeth 603 of the cam 650. In some embodiments, the disk 692 defines an opening 693 through which the cam 650 can be fixedly coupled with a lever that is actuatable from an exterior of the housing. The stopper 604 can be fixed relative to the housing. In some embodiments, the stopper 604 is integrally formed with the housing. In other embodiments, the stopper 604 is a separate component that is fixedly attached to the housing.

With reference to FIG. 21, the illustrated linkage 691 includes a channel 697 through which a rod or other pivotal member can be inserted to pivotably couple the linkage 691 to the trigger 642. The linkage 691 can further include a wedge 695, which may also be referred to as a deflector. In some embodiments, the wedge 695 includes a cutout or recess 696, which may accommodate an axle, rod, or other pivotal member to which the cam is coupled for purposes of rotation. In some embodiment, a portion of a lever (such as the lever 118), or of a linkage to the lever, by which the cam is actuated, can pass through the recess 696. The lever can function with the cam in manners such as previously discussed.

With reference again to FIG. 18, operation of the cam-locking mechanism 640 will now be described. In the illustrated embodiment, the deflection member 690 comprises either a separate component that is fixedly secured to the housing or is integrally formed with the housing. The deflection member 690 is schematically depicted as a cube, but a variety of suitable shapes and configurations are contemplated. The deflection member 690 includes an angled deflection surface 699 configured to interact with the wedge 695 of the linkage 691.

When the trigger 642 is depressed, the linkage 691 is advanced inwardly and the wedge 695 is deflected by the deflection surface 699. This, in turn, displaces the cam 650 laterally toward the stopper 644. When the trigger 642 is depressed by a sufficient amount the teeth cam and the stopper 644 fully engage and prevent rotation of the cam 650 relative to the housing.

Accordingly, in the illustrated embodiment, the teeth 603, 605 can grip on the side (e.g., lateral surface) of the cam 650. As with other teeth-engaging embodiments described above, as with respect to FIG. 14, the locking mechanism can be a binary system, rather than a variable friction system. In some instances, employing relatively smaller and/or more numerous teach can reduce the possibility and/or severity of trigger-action-induced rotation of the cam. In some instances, relatively larger teeth may require less trigger force to lock the cam or to maintain the cam in the locked state. In further instances, these and/or other characteristics of differently sized teeth may be balanced to achieve a desired engagement force with minimal ancillary cam rotation.

In some embodiments, releasing the trigger 642 is sufficient to permit the cam 650 to rotate again, when the lever (e.g., the lever 118) is rotated. The angled teeth (substantially triangularly shaped, in the illustrated embodiment) can automatically urge the cam 650 away from the stopper 644 when the lever is rotated. The wedge 695 may similarly be forced outward to retract the trigger 642 to an undepressed configuration. In other embodiments, the linkage 691 and/or the trigger 642 may be biased to the undepressed orientation in manners such as previously discussed.

In other embodiments, the interlocking teeth may be replaced with frictionally engaging surfaces, including one or more of the various frictional members discussed elsewhere herein. In other or further embodiments, the cam 650 may include a series of holes or depressions that extend transversely therethrough or therein, and the stopper 644 can include a series of complementary posts or pins sized to fit within the holes or depressions. The locking mechanism can be selectively deactivated to remove the posts or pins from the cam 650, thereby freeing the cam 650 to again rotate relative to the housing. In other embodiments, the arrangement of posts and holes may be reversed.

In the illustrated embodiment, a wedge is used to generate the transverse motion that engages the cam to the stopper. In other embodiments, any suitable linkage, lever, etc. could also or alternatively be used to create the transverse motion.

In some embodiments, the trigger 642 is positioned at a lateral side of the housing, rather than on the front of the housing. This may also be the case with other triggers disclosed elsewhere herein.

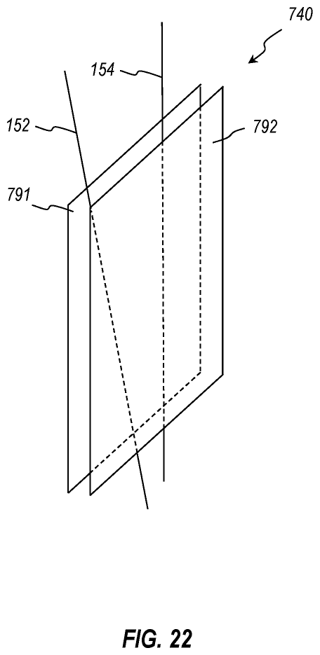

FIG. 22 depicts another embodiment of a locking mechanism 740 that is configured to lock the distal end of the shaft in a fixed angular deflection pattern. In this embodiment, the locking mechanism 7410 acts directly on the tensioning lines 152, 154. As with other embodiments, variable friction is achievable. The trigger may be spring loaded (e.g., for biasing to an unactuated or disengaged state).

In the illustrated embodiment, a stationery friction pad 791 is placed in light contact with the tensioning lines 152, 154, and a different friction 792 pad is attached to a laterally deflecting trigger mechanism, such as, for example, a mechanism such as the trigger 642/linkage 691/deflection surface 699 system described above with respect to FIGS. 18-21. The second friction pad 692 can be placed on the other side of the tensioning lines 152, 154 with enough clearance from the stationary pad 791 for the tensioning lines 152, 154 to pass freely between the pads 791, 792. When the trigger is pulled, the trigger mechanism friction pad 792 is pressed into the stationary pad 791 with the tensioning lines 152, 154 trapped between them, thus creating friction on the tensioning lines. The friction may be sufficient to prevent movement of the tensioning lines. In various embodiments, at least one friction pad 791, 792 directly engages one or more of the tensioning liens 152, 154 to maintain the same in fixed relation relative to the handle.

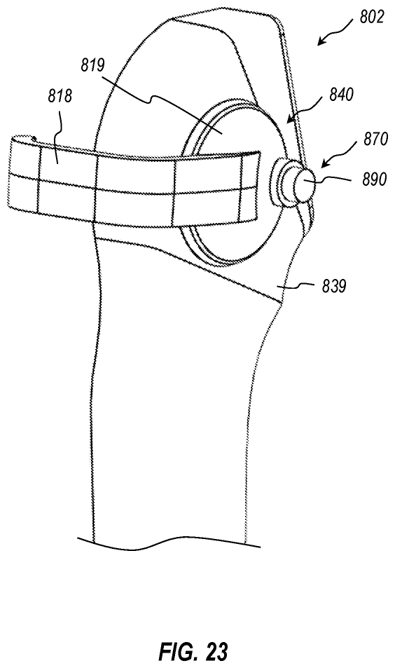

FIGS. 23-26 illustrate another embodiment of an endoscope 802 that includes a locking mechanism 840. The locking mechanism 840 is configured to selectively fix a lever 818 relative to a housing 839. The lever 818 is fixedly coupled to a tensioning cam at an interior of the housing 839, such as tensioning cams previously disclosed. Accordingly, by fixing the lever 818 relative to the housing 839, the tensioning cam is likewise rotationally fixed relative to the housing 839.

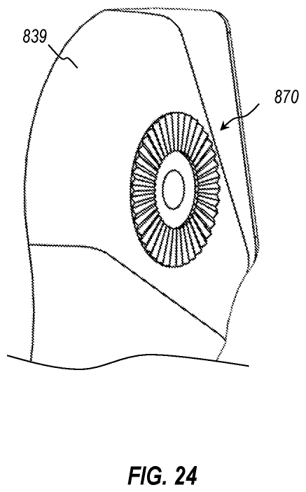

In the illustrated embodiment, the lever 818 includes a hood 819 configured to encompass an array of teeth 870 that are fixedly secured to the housing 839, as depicted in FIG. 24. The teeth 870 may be part of a separate component that is secured to the housing 839 or may be integrally formed therewith.

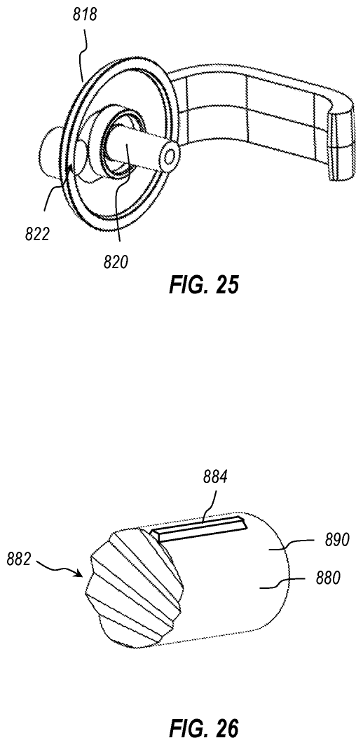

With reference to FIG. 25, the lever 818 can include a projection 820 that is configured to pass through an opening at the center of the array of teeth 870 and into the housing 839. The projection 820 can be fixedly secured to the tensioning cam. The lever 818 can further include a channel 822 that is configured to receive at least a portion of a two-state locking system 870 that selectively locks a locking element 880 (FIG. 26) in proximity to the array of teeth 870 and selectively permits the locking element 880 to retract from the teeth to unlock the locking mechanism 840 and permit free movement of the lever 818.

With reference to FIG. 26, the locking element 880 can include an array of teeth 882 that is complementary to and capable of interlocking with the array of teeth 870 defining by the housing 839.

With continued reference to FIG. 26 and reference again to FIG. 23, the locking element 880 can be moved via an actuator 890, which may be accessible at an exterior of the lever 818. In the illustrate embodiment, the actuator 890 is integrally formed with the locking element 880. The actuator 890 may also be referred to as a locking actuator.

With reference again to FIGS. 25 and 26, in some embodiments, the channel 822 and the locking element 880 are keyed to each other to maintain a fixed angular relationship. This can ensure proper alignment of the teeth 882 with the teeth 870, in some instances. In the illustrated embodiment, the locking element 880 includes one or more longitudinal projections 884 that are configured to fit within a complementary longitudinal groove defined by the channel 822. Any other suitable keying arrangement is contemplated.

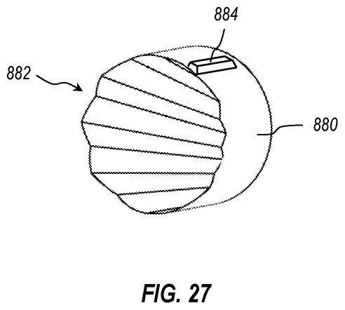

Any suitable method for selectively maintaining the locking element 880 in an advanced configuration to lock with the teeth 870 is contemplated. In one embodiment, a shorter locking element 880 is used, such as that depicted in FIG. 27. Additional components are interconnected to the locking element 880 and the channel 822 of the lever 818 to form a two-state click lock system. Such two-state click lock systems are well known in the art and can be readily adapted to function with the locking element 880 and the channel 822 of the lever 818. Such a system advances the locking element 880 into interlocking engagement with the teeth 870 of the housing 839, such as by pushing on a button that is interconnected with the locking element 880, and maintains this locked configuration while the system remains in a locked state. The system can be transitioned to the unlocked state by again pushing on the button, which retracts the locking element 880 from the teeth 870.

Illustrative examples of two-state click lock systems include a variety of such systems that are used with retractable pens. For example, a number of suitable two-state click lock systems that can be used with the locking mechanism 840 include those disclosed in U.S. Pat. No. 3,205,863, titled WRITING INSTRUMENT, issued Sep. 14, 1965, the entire contents of which are incorporated by reference herein. As just one example, the embodiment depicted in FIGS. 16-20 of the '863 patent and described with respect thereto may be suitably incorporated into the locking mechanism 840. Rather than selectively advancing and retracting an ink cartridge and ball-point pen, the locking mechanism therein disclosed would instead selectively advance and retract the locking element 880.

In other embodiments, rather than an interlocking teeth system, the locking mechanism can use frictional engagement mechanisms such as previously disclosed. For example, a friction pad may be used in place of the teeth 870 and/or a friction pad may be used in place of the teeth 882.

In some embodiments, the locking mechanism 840 is located on a top of the lever 818.

Previously known single-use ureteroscopes suffer from a variety of drawbacks. In some instances, the ureteroscopes have a low-resolution camera. In other or further instances, the handle heats up during use. In other or further instances, the distal tip position must be held manually throughout a procedure, such as by maintaining contact with an actuator for deflecting the distal tip. In other or further instances, the proximal portions of the shafts are insufficiently stiff for desired pushability within the patient.

Various embodiments disclosed herein address, ameliorate, and/or remedy one or more of the limitations listed in the previous paragraph. In certain embodiments, the endoscope includes an articulation lock, which can maintain a desired position of the deflected distal tip during a procedure. In other or further embodiments, the distal camera can have an enhanced resolution. In other or further embodiments, the endoscope can have a lower overall cost. In some embodiments, the endoscope includes auto-adjust lighting with cool LEDs. In still other or further embodiments, the endoscope includes a relatively stiff proximal shaft for pushability and torqueing, and further includes a softer distal shaft region for passive deflection.

In one illustrative example, a ureteroscope can include a camera having a 160,000-pixel resolution, a working distance focus of from 2 to 50 millimeters, and field of view of 120 degrees. The handle includes integrated video and picture capture capabilities. The lights are auto adjusting. The proximal shaft size is 8.95 French. The distal tip is 7.5 French. The working channel size is 3.6 French. The deflection angle in either direction is 270 degrees. The ureteroscope includes a location-holding brake mechanism, or a locking mechanism, such as any of those previously described.

The claims following this written disclosure are hereby expressly incorporated into the present written disclosure, with each claim standing on its own as a separate embodiment. This disclosure includes all permutations of the independent claims with their dependent claims. Moreover, additional embodiments capable of derivation from the independent and dependent claims that follow are also expressly incorporated into the present written description. These additional embodiments are determined by replacing the dependency of a given dependent claim with the phrase “any of the preceding claims up to and including claim [x],” where the bracketed term “[x]” is replaced with the number of the most recently recited independent claim. For example, for the first claim set that begins with independent claim 1, claim 3 can depend from either of claims 1 and 2, with these separate dependencies yielding two distinct embodiments; claim 4 can depend from any one of claim 1, 2, or 3, with these separate dependencies yielding three distinct embodiments; claim 5 can depend from any one of claim 1, 2, 3, or 4, with these separate dependencies yielding four distinct embodiments; and so on.

Recitation in the claims of the term “first” with respect to a feature or element does not necessarily imply the existence of a second or additional such feature or element. Embodiments of the invention in which an exclusive property or privilege is claimed are defined as follows.

1. An endoscope comprising:
an elongated shaft comprising a distal tip, the shaft comprising a displaceable distal region;
a handle coupled to the elongated shaft;
a tensioning actuator movable relative to the handle;
at least one tensioning line extending through at least a proximal portion of the elongated shaft and through a portion of the handle, the at least one tensioning line being coupled to the displaceable distal region of the shaft and further being coupled to the tensioning actuator such that movement of the tensioning actuator relative to the handle effects angular movement of the displaceable distal region; and
a locking mechanism configured to prevent angular movement of the displaceable distal region when the locking mechanism is in an engaged state.

2-5. (canceled)

6. The endoscope of claim 1, wherein the locking mechanism is biased to a disengaged state, and wherein the bias must be overcome to transition the locking mechanism to the engaged state.

7. The endoscope of claim 6, further comprising a biasing member that biases the locking mechanism to the disengaged state, wherein the biasing member is configured to automatically return the locking mechanism to the disengaged state.

8. The endoscope of claim 7, wherein the locking mechanism further comprises a manually actuatable release actuator configured to transition the locking mechanism from the engaged state to the disengaged state in the event that the biasing member fails to automatically return the locking mechanism to the disengaged state.

9. The endoscope of claim 6, wherein the locking mechanism further comprises a release actuator configured to transition the locking mechanism from the engaged state to the disengaged state.

10. The endoscope of claim 6, wherein the locking mechanism comprises a spring that biases the locking mechanism to the disengaged state.

11. The endoscope of claim 1, further comprising a cam coupled to the lever and to the at least one tensioning line, wherein the locking mechanism fixes an angular orientation of the cam relative to the housing when the locking mechanism is engaged.

12. The endoscope of claim 11, wherein the locking mechanism is moved into frictional contact with the cam to fix the angular orientation of the cam relative to the housing when the locking mechanism is engaged.

13. The endoscope of claim 11, wherein the locking mechanism comprises a wedged stopper that is pressed into frictional contact with both the cam and the handle when the locking mechanism is engaged.

14-18. (canceled)

19. An endoscope comprising:
an elongated shaft comprising a distal tip, the shaft comprising a displaceable distal region;
a handle coupled to the elongated shaft;
a tensioning cam movable relative to the handle;
at least one tensioning line extending through at least a proximal portion of the elongated shaft and through a portion of the handle, the at least one tensioning line being coupled to the displaceable distal region of the shaft and further being coupled to the tensioning cam such that movement of the tensioning cam relative to the handle effects angular movement of the displaceable distal region; and
a locking mechanism comprising a trigger at a forward side of the handle, the trigger being configured to be actuated by a finger of a hand of a user while the hand holds the handle, wherein actuation of the trigger comprises depressing the trigger inwardly relative to the handle, and wherein when the trigger is depressed to a locked state, the trigger is interconnected with the tensioning cam so as to prevent rotational movement of the tensioning cam and thereby prevent angular movement of the displaceable distal region of the elongated shaft.

20. The endoscope of claim 19, wherein further comprising a biasing member that provides a bias to automatically transition the trigger to an unlocked state.

21. The endoscope of claim 20, wherein when the trigger is released from the locking state, the biasing member automatically urges the trigger away from the longitudinal axis to the unlocked state.

22. The endoscope of claim 20, wherein the bias provided by the biasing member must be continually overcome to maintain the trigger in the locked state.

23. The endoscope of claim 20, further comprising a trigger lock configured to maintain the trigger in the locked state when the trigger lock is actuated to a holding state.

24. The endoscope of claim 19, wherein actuation of the trigger comprises depressing the trigger toward a longitudinal axis of the handle.

25. The endoscope of claim 19, further comprising a linkage that interconnects the trigger with the tensioning cam.

26. The endoscope of claim 25, wherein the linkage comprising a wedge.

27. The endoscope of claim 26, wherein the wedge directly contacts the tensioning cam.

28. The endoscope of claim 26, wherein the wedge is configured to move the tensioning cam transversely along an axis of rotation of the tensioning cam.

29. The endoscope of claim 26, wherein the wedge is configured to contact a lateral surface of the tensioning cam.

30-33. (canceled)
